(19) United States
(12) Patent Application Publication
(10) Pub. No.: US 2019/0153512 A1
(43) Pub. Date: May 23, 2019

(54) Methods for Enriching Microbial Cell-Free DNA in Plasma

(71) Applicants: THE TRANSLATIONAL GENOMICS RESEARCH INSTITUTE, PHOENIX, AZ (US); THE ARIZONA BOARD OF REGENTS ON BEHALF OF THE UNIVERSITY OF ARIZONA, TUCSON, AZ (US)

(72) Inventors: Muhammed MURTAZA, Phoenix, AZ (US); Mehreen KISAT, Tucson, AZ (US); Ahuva ODENHEIMER-BERGMAN, Phoenix, AZ (US)

(73) Assignees: THE TRANSLATIONAL GENOMICS RESEARCH INSTITUTE, Phoenix, AZ (US); The ARIZONA BOARD OF REGENTS ON BEHALF OF THE UNIVERSITY OF ARIZONA, TUCSON, AZ (US)

(21) Appl. No.: 16/197,319

(22) Filed: Nov. 20, 2018

Related U.S. Application Data
(60) Provisional application No. 62/588,782, filed on Nov. 20, 2017.

(51) Int. Cl. C12Q 1/6806; C12Q 1/6869

(52) U.S. Cl. CPC C12Q 1/6806; C12Q 1/6869

(57) ABSTRACT

Methods are provided for detecting non-human candidate DNA within a plasma sample from a human subject. A method of diagnosing and characterizing a bacterial infection may include the steps of obtaining a plasma sample from a subject suspected of having a bacterial infection, extracting cell-free DNA (cfDNA) from the plasma sample, performing whole genome sequencing on the cfDNA to obtain sequencing data, aligning the sequencing data with a human genome to identify human DNA and non-human DNA, removing the human DNA from the sequencing data, assigning the non-human DNA to a candidate pathogen DNA, selecting a subset of the non-human DNA based on a fragment length of the non-human DNA, and determining the presence of the candidate pathogen DNA within the subset of the non-human DNA.

### CROSS-REFERENCE TO RELATED APPLICATIONS

This application claims the benefit of U.S. Provisional Patent Application No. 62/588,782, filed on Nov. 20, 2017, the contents of which is incorporated herein by reference in its entirety.

### STATEMENT REGARDING FEDERALLY SPONSORED RESEARCH

This invention was made with governmental support under grant number SU2C-AACR-PS-14 awarded by the American Association for Cancer Research (AACR). The United States government has certain rights in the invention.

### FIELD

The present invention relates to the field of methods of characterizing a patient's microbiome for treatment of the patient based on the characterized microbiome, and more specifically, to using whole genome plasma DNA sequencing for diagnosis and treatment of infection and disease.

### BACKGROUND

The ability to use blood to diagnose and monitor disease has been a pillar of modern medicine. In patients with infection or sepsis, identification of the pathogen causing the infection or sepsis may be performed using conventional microbiology approaches such as blood culture and urine culture. These conventional culturing approaches take at least 4-5 days to effectively identify the pathogen responsible for causing sepsis. Moreover, sensitivity of blood culture is estimated at approximately 30%.

With the advent of high-throughput nucleic acid sequencing, the analysis of blood has been extended to the study of cell-free DNA (cfDNA). cfDNA analysis has found utility in diagnostic applications, for example in cancer diagnostics. However, the amounts of cfDNA available in a sample are generally very limited, and sampling and process inefficiencies of current methods further limit the effective amount of cfDNA available to analyze.

### SUMMARY

A need exists for methods of identifying microbes and/or pathogens in patients, for example in patients with sepsis and/or cancer, and for predicting a blood culture result using non-invasive methods. In clinical applications, for example, a need exists to distinguish between post-transplant infection and organ rejection. The present invention employs whole genome sequencing (WGS) of plasma DNA to detect and identify microbes, pathogens, and commensal organisms in a blood or plasma sample. In one embodiment, direct sequencing of bacterial DNA in plasma is feasible and may allow rapid identification of pathogens in patients with sepsis.

Methods are provided herein for diagnosing a pathogen in a plasma sample. In various embodiments, the method may comprise the steps of obtaining the plasma sample from a subject suspected of having the pathogen, extracting cell-free DNA (cfDNA) from the plasma sample, selecting a subset of the cfDNA based on the size of the cfDNA, performing whole genome sequencing on the subset of the cfDNA to obtain sequencing data, assigning the sequencing data to a candidate pathogen DNA, and determining a presence of the pathogen in the plasma sample.

Methods are provided herein for detecting a microbe in a plasma sample. In various embodiments, the method may comprise the steps of obtaining the plasma sample from a subject, and extracting cfDNA from the plasma sample. The extracted cfDNA may comprise human cfDNA and non-human cfDNA. The method may further comprise the steps of determining a size threshold associated with human cfDNA, selecting a subset of the extracted cfDNA based on the subset having a size below the size threshold, performing whole genome sequencing on the subset of the extracted cfDNA to obtain sequencing data, assigning the sequencing data to a candidate microbe DNA, and determining a presence of the microbe in the plasma sample.

Methods are provided herein for detecting a microbe in a plasma sample. In various embodiments, the method may comprise the steps of obtaining the plasma sample from a subject, and extracting cfDNA from the plasma sample. The extracted cfDNA may comprise human cfDNA and non-human cfDNA. The method may further comprise the steps of determining a fragment length threshold associated with human cfDNA, performing whole genome sequencing on the extracted cfDNA to obtain sequencing data for the human cfDNA and the non-human cfDNA, selecting a subset of the sequencing data based on the subset having a sequencing read length below the fragment length threshold, assigning the subset of the sequencing data to a candidate microbe DNA, and determining a presence of the microbe in the plasma sample.

Methods are provided herein for detecting non-human candidate DNA within a plasma sample from a human subject. In various embodiments, the method may comprise the steps of obtaining the plasma sample from the human subject, and extracting cfDNA from the plasma sample. The extracted cfDNA may comprise human cfDNA and non-human cfDNA. The method may further comprise the steps of determining a size threshold associated with human cfDNA, selecting a subset of the extracted cfDNA based on the subset having a size below the size threshold, performing whole genome sequencing on the subset of the extracted cfDNA to obtain sequencing data, and assigning the sequencing data to a non-human candidate DNA.

Methods are provided herein for enriching non-human cfDNA within a sample from a human subject. In various embodiments, the method may comprise the steps of obtaining the sample from the human subject, and extracting cfDNA from the sample. The extracted cfDNA may comprise human cfDNA and non-human cfDNA. The method may further comprise the steps of determining a size threshold associated with human cfDNA, and selecting a subset of the extracted cfDNA based on the subset having a size below the size threshold. The subset may comprise a greater ratio of non-human cfDNA to human cfDNA than the extracted cfDNA.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the figures, wherein like numerals may denote like elements.

FIG. 1 illustrates a schematic of the approach used for validating the disclosed methods;

FIG. 2 illustrates a block diagram of the clinical study for the disclosed methods;

FIG. 3 illustrates a schematic of the sequencing approach and analysis used for the disclosed methods;

FIG. 4 illustrates a graph of DNA density versus DNA fragment size for three pathogens;

FIG. 5 illustrates results of whole genome plasma DNA sequencing where the patient's blood culture was negative for the pathogens;

FIG. 6 illustrates results comparing the fraction of bacterial reads from raw sequencing data to the fraction of bacterial reads after size selection is applied to the sequencing data; and

FIG. 7 illustrates results of size-selection enrichment for increasing the fraction of sequencing reads successfully classified as bacterial.

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

Circulating cell-free DNA (cfDNA) is comprised of short extracellular DNA fragments (ranging from approximately 160 to 180 base pairs) found in body fluids such as plasma or urine. cfDNA in human bodily fluids carries non-human DNA from microbes and pathogens in addition to a substantial proportion of human DNA. For example, a human plasma sample may contain, in addition to human cfDNA, cfDNA of one or more commensal bacteria as well as cfDNA from one or more infection-causing microbes or pathogens, such as a pathogenic bacteria. In patients with cancer, a variable fraction of cfDNA in plasma is contributed by cancer cells. These DNA fragments, known as circulating tumor DNA (ctDNA), carry tumor-specific somatic genetic alterations. Analysis of circulating cfDNA from plasma has several potential diagnostic applications in transplant and cancer medicine.

Sequencing cfDNA in plasma and other body fluids can rapidly identify pathogens by classifying non-human sequencing reads to microbes and potential pathogens. However, greater than (>) 98% of cfDNA in circulation originates from human cells, making previous approaches for pathogen identification expensive and time-consuming.

cfDNA is predominantly understood to result from enzymatic degradation during or after cell death as apoptotic cells release nucleosome-protected DNA fragments into the circulation. The half-life of cfDNA is estimated to be approximately 2 hours. Analysis of cfDNA can be affected by many technical factors that must be considered when evaluating plasma genotyping results including limited amounts of fragmented cfDNA, variable tumor fractions in cfDNA across patients, sampling inefficiencies in previous analytical methods, pre-analytical variables such as time between blood collection and sample processing and background noise affecting reliability of low-abundance mutations.

As discussed, human cfDNA in plasma predominantly exists as 160-180 bp fragments because mono-nucleosomal fragments protect DNA from further degradation. The inventors investigated the relative size of circulating microbial DNA (microbial cfDNA) and found that microbial cfDNA fragments in plasma are shorter in length than human cfDNA fragments in plasma, because prokaryotic DNA is not wrapped into nucleosomes. Pair-end sequencing was performed to determine with high confidence that DNA fragment size of microbial cfDNA was smaller than the fragment size of human plasma cfDNA. The inventors have determined that this size difference enables size selection and enrichment of non-human DNA and potentially increase the yield of microbial cfDNA from plasma samples. The disclosed method of size selection to enrich for non-human DNA in plasma will expand the applications of whole genome sequencing from cfDNA in plasma, urine and other body fluids for indications such as sepsis and microbiome analysis in cancer patients. The presently disclosed approach will lower costs of sequencing, reduce turnaround time and increase on target rates and sensitivity. The presently disclosed approach may enable delineation of antibiotic resistance by increasing the coverage of microbial DNA in plasma samples.

The sample in this method is preferably a biological sample from a subject. The term “sample” or “biological sample” is used in its broadest sense. Depending upon the embodiment of the invention, for example, a sample may comprise a bodily fluid including whole blood, serum, plasma, urine, saliva, cerebral spinal fluid, semen, vaginal fluid, pulmonary fluid, tears, perspiration, mucus and the like; an extract from a cell, chromosome, organelle, or membrane isolated from a cell; a cell; genomic DNA, RNA, or cDNA, in solution or bound to a substrate; a tissue; a tissue print, or any other material isolated in whole or in part from a living subject or organism. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues.

In some embodiments, sample or biological sample may include a bodily tissue, fluid, or any other specimen that may be obtained from a living organism that may comprise additional living organisms. By way of example only, in some embodiments, sample or biological sample may include a specimen from a first organism (e.g., a human) that may further comprise an additional organism (e.g., bacteria, including pathogenic or non-pathogenic/commensal bacteria, viruses, parasites, fungi, including pathogenic or non-pathogenic fungi, etc.). In some embodiments of the invention, the additional organism may be separately cultured after isolation of the sample to provide additional starting materials for downstream analyses. In some embodiments, the sample or biological sample may comprise a direct portion of the additional, non-human organism and the host organism (e.g., a biopsy or sputum sample that contains human cells and fungi).

With respect to use of the sample or biological sample, embodiments of the claimed methodology provide improvements compared to conventional methodologies. Specifically, conventional methodologies of identifying and characterizing microorganisms include the need for morphological identification and culture growth. As such, conventional methodologies may take an extended period of time to identify the microorganism and may then require further time to identify whether the microorganism possesses and certain markers. Some embodiments of the invention can provide a user with information about any microorganisms present in a sample without the need for additional culturing because of the reliance of nucleic acid amplification and sequencing. In other words, direct extraction of nucleic acids coupled with amplification of the desired markers and downstream sequencing can reduce significantly the time required to obtain diagnostic and strain identifying information.

The term “extraction” as used herein refers to any method for separating or isolating the nucleic acids from a sample, more particularly from a biological sample, such as blood or plasma. Nucleic acids such as RNA or DNA may be released, for example, by cell lysis. Moreover, in some aspects, extraction may also encompass the separation or isolation of extracellular RNAs (e.g., extracellular miRNAs) from one or more extracellular structures, such as exosomes.

Some embodiments of the invention include the extraction of one or more forms of nucleic acids from one or more samples. In some aspects, the extraction of the nucleic acids can be provided using one or more techniques known in the art. In other embodiments, methodologies of the invention can use any other conventional methodology and/or product intended for the isolation of intracellular and/or extracellular nucleic acids (e.g., DNA or RNA).

The term “nucleic acid” or “polynucleotide” as referred to herein comprises all forms of RNA (mRNA, miRNA, rRNA, tRNA, piRNA, ncRNA), DNA (genomic DNA, mtDNA, cfDNA, ctDNA), as well as recombinant RNA and DNA molecules or analogs of DNA or RNA generated using nucleotide analogues. The nucleic acids may be single-stranded or double-stranded. The nucleic acids may include the coding or non-coding strands. The term also comprises fragments of nucleic acids, such as naturally occurring RNA or DNA which may be recovered using one or more extraction methods disclosed herein. “Fragment” refers to a portion of nucleic acid (e.g., RNA or DNA).

As used herein, a “whole genome sequence”, or WGS (also referred to in the art as a “full”, “complete”, or entire” genome sequence), or similar phraseology is to be understood as encompassing a substantial, but not necessarily complete, genome of a subject. In the art the term “whole genome sequence” or WGS is used to refer to a nearly complete genome of the subject, such as at least 95% complete in some usages. The term “whole genome sequence” or WGS as used herein does not encompass “sequences” employed for gene-specific techniques such as single nucleotide polymorphism (SNP) genotyping, for which typically less than 0.1% of the genome is covered. The term “whole genome sequence”, or WGS as used herein does not require that the genome be aligned with any reference sequence, and does not require that variants or other features be annotated. As used herein the term “whole genome sequencing” refers to determining the complete DNA sequence of the genome at one time.

The term “library,” as used herein refers to a library of genome/transcriptome-derived sequences. The library may also have sequences allowing amplification of the “library” by the polymerase chain reaction or other in vitro amplification methods well known to those skilled in the art. In various embodiments, the library may have sequences that are compatible with next-generation high throughput sequencing platforms. In some embodiments, as a part of the sample preparation process, “barcodes” may be associated with each sample. In this process, short oligonucleotides are added to primers, where each different sample uses a different oligo in addition to a primer.

In certain embodiments, primers and barcodes are ligated to each sample as part of the library generation process. Thus during the amplification process associated with generating the ion amplicon library, the primer and the short oligo are also amplified. As the association of the barcode is done as part of the library preparation process, it is possible to use more than one library, and thus more than one sample. Synthetic nucleic acid barcodes may be included as part of the primer, where a different synthetic nucleic acid barcode may be used for each library. In some embodiments, different libraries may be mixed as they are introduced to a flow cell, and the identity of each sample may be determined as part of the sequencing process.

The following examples are given for purely illustrative and non-limiting purposes of the present invention.

### Example 1

The disclosed methods and analyses show that DNA from pathogens of sepsis is detectable in plasma DNA, and that whole genome sequencing (WGS) with size selection and outlier detection can be used to identify etiology of sepsis.

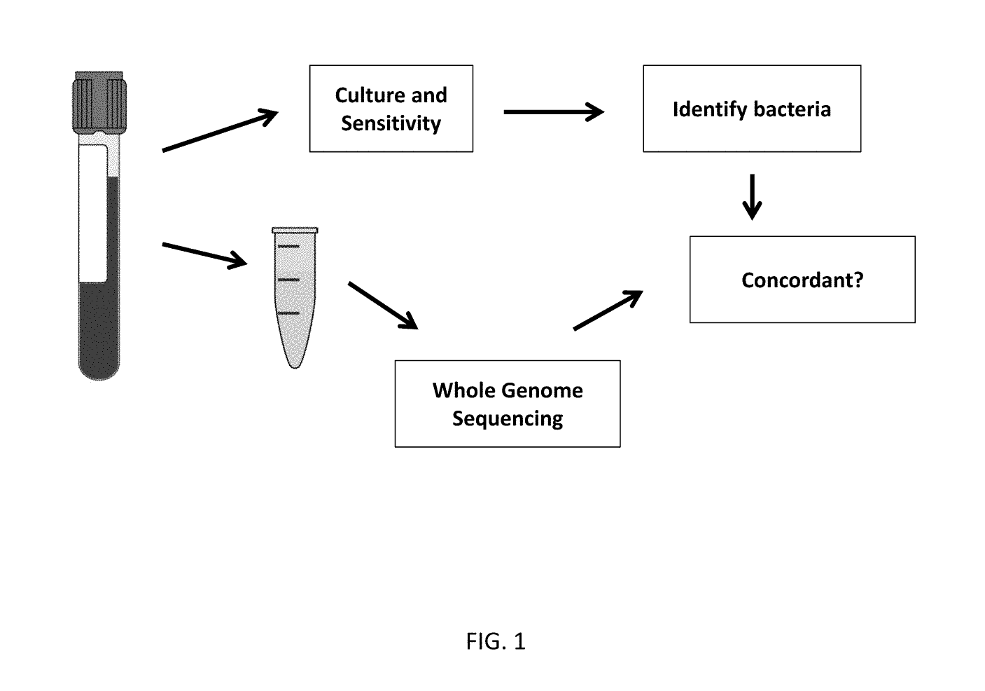

FIG. 1 shows the approach used for this study. To validate the approach, patients with sepsis were evaluated in a clinical setting, where the present WGS method could be compared to conventional culturing methods. In patients with sepsis, microbial DNA was detectable in plasma using the disclosed method, enabling rapid identification and characterization of antimicrobial sensitivity.

### Methods

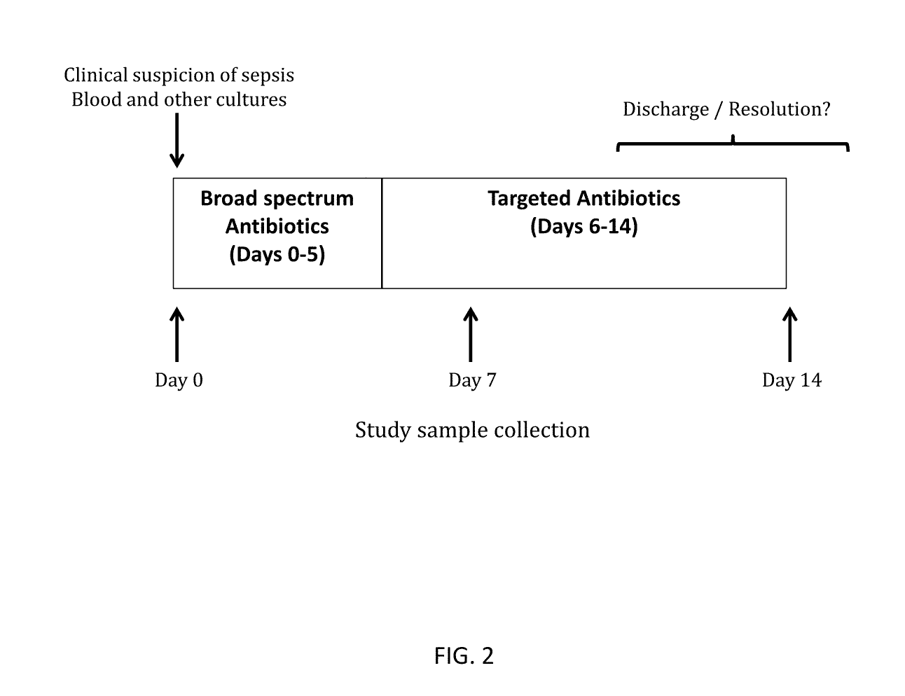

FIG. 2 shows the clinical study outline and timeline. For this study, thirty (30) consecutive patients in critical care suspected of sepsis were enrolled. The patients (also referred to as subjects) were 18 years of age or older, with systemic inflammatory response syndrome and with a clinical suspicion of sepsis, and were clinically prescribed a blood panel with culture.

Plasma samples from the thirty (30) subjects were collected at the time of diagnostic workup for bacterial sepsis. Three (3) serial plasma samples were taken for each subject, the first sample taken at day zero (0), the second sample taken at day seven (7), and the third sample taken at day (14). Plasma samples were collected on the same day when blood was drawn for cultures. Plasma collected at first time point from thirty (30) patients was whole genome sequenced according to the following methods.

For WGS sample preparation, the one or two cell-free DNA BCT (Streck) tubes collected from each subject were processed within 24 hours after collection. Samples were centrifuged at 820 g for 10 minutes at room temperature. Five 1 milliliter (mL) aliquots of plasma were further centrifuged at 16,000 g for 10 minutes to pellet any remaining cellular debris. The supernatant was stored at −80° C. until DNA extraction.

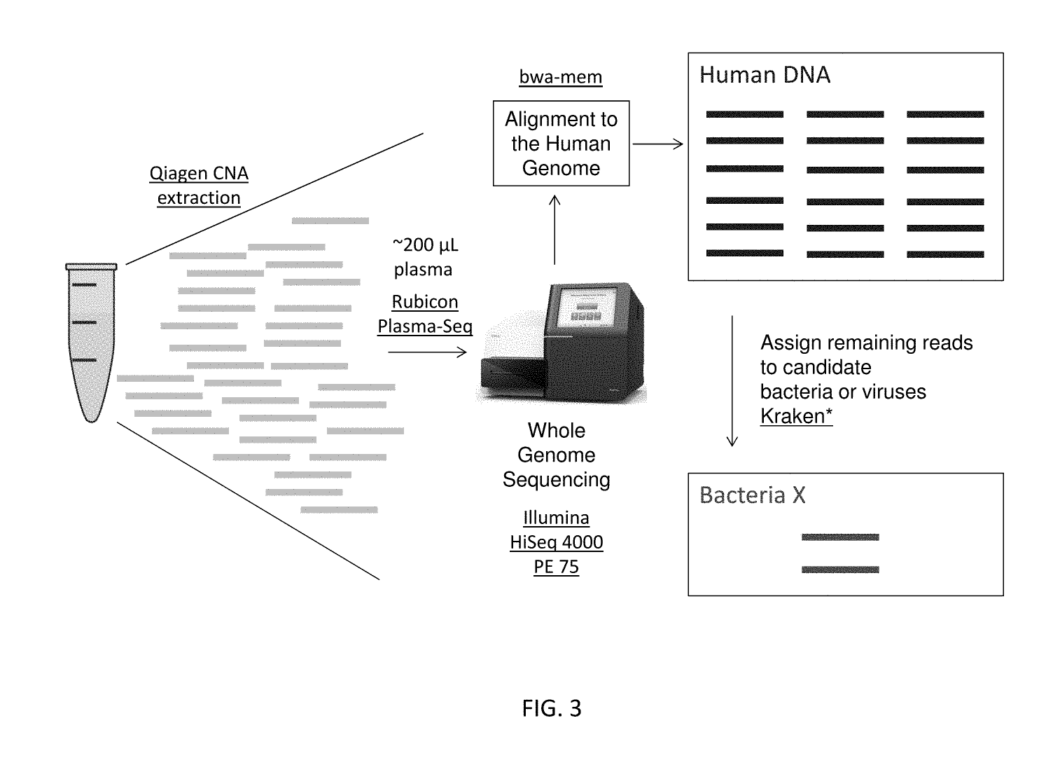

FIG. 3 shows the sequencing approach and analysis method. After collection of the biological samples, i.e., the plasma samples, the extraction of cfDNA from the samples was performed using QIAGEN® Circulating Nucleic Acid (CAN) extraction kit.

Whole genome sequencing libraries were prepared using Rubicon Plasma-Seq. Whole genome sequencing was performed using Illumina HiSeq 4000. In one embodiment, between approximately 136 million and 220 million reads per sample were obtained by whole genome sequencing. In another embodiment, between approximately 14 million and 42 million reads per sample were obtained by whole genome sequencing. The number of reads per sample at this stage represents the raw number of sequencing reads prior to the size selection steps.

Sequencing reads were then aligned to the human genome using the BWA-MEM alignment algorithm. As expected, greater than (>) 98% of the reads aligned to the human genome. Thus, the subset of reads that aligned with the human genome was a large proportion of the raw sequencing reads. The human DNA reads were then removed or subtracted from the data set to produce a subset of reads which were unmapped to the human genome. At least a portion of the remaining unmapped reads were expected to be non-human, e.g. bacterial, viral, or fungal. After subtracting the human DNA reads to produce the subset of unmapped reads, an informatics approach was used to identify sources of the non-human DNA. The non-human DNA was then evaluated to assign the unmapped reads to a list of candidate bacteria or viruses. Of the unmapped reads, 0.69%-50% classified to RefSeq genomes from bacteria or viruses (median 2.4%).

### Results

TABLE 1 shows results of cultures from clinical samples co-incident with plasma samples taken at the first time point (day 0). Of the total cultures performed, the column titled “Growth” shows the positive cultures. For example, three (3) of the fifty (50) blood cultures performed for the thirty (30) patients showed positive culture results.

TABLE 1
Cultures from clinical samples collected at day 0

| | | | Pathogen Identified |
|---|---|---|---|
| | Total | Growth | in Culture |
| Blood | 50 | 3 | 3 |
| Urine | 7 | 4 | 3 (plus 1 fungal |
| | | | pathogen) |
| Broncheo-alveolar | 5 | 5 | 4 |
| Lavage (BAL) | | | |
| Peritoneal fluid | 3 | 1 | 1 |
| Sputum | 3 | 3 | 1 |
| Stool | 2 | 0 | 0 |
| CSF | 1 | 0 | 0 |

Three (3) of the thirty (30) patients with sepsis had positive blood cultures growing Escherichia coli (E. coli), Group B Streptococcus, and Staphylococcus haemolyticus respectively. The culture results were used to validate the disclosed size-selected WGS plasma sequencing method. For the three samples with positive blood cultures, and one healthy control, 80-120 million WGS reads per sample were generated. As expected, 95-98% of sequencing reads were of human origin. When ranked by number of informative reads, the expected bacterial species seen on blood culture was enriched and ranked 1/97, 7/307 and 4/55 candidates in patient samples. Corresponding ranks in the control sample were 119, 63 and 14 of 328 candidates.

TABLE 2 shows the WGS read results from plasma samples taken from the three (3) patients which showed positive blood culture results. One patient (KSEP-013) had a positive culture result for E. coli, one patient (KSEP-020) had a positive culture result for Group B Streptococcus, and one patient (KSEP-033) had a positive culture result for Staphylococcus haemolyticus. The genus level and species level reads are shown for each patient plasma sample, as well as the percent (%) of the species found within the classified reads. The Z-score represents the comparison of the organism within the sample as compared to that organism within the other 29 samples. For example, in patient KSEP-020, the reads for Group B Streptococcus had a Z-score of 5.6595, which is approximately five (5) standard deviations away from the population mean.

TABLE 2
WGS reads for organisms isolated from Blood Cultures

| | | | | % Species | Z-score |
|---|---|---|---|---|---|
| | | Genus | Species | within | before |
| | Organism on | Level | Level | Classified | Size |
| Patient ID | Culture | Reads | Reads | Reads | Selection |
| KSEP-013 | E. coli | 163 | 143 | 0.004% | −0.5277 |
| KSEP-020 | Group B | 268 | 236 | 0.04% | 5.6595 |
| | Streptococcus | | | | |
| — | Epstein-Barr | 758 | 754 | 0.13% | 1.2134 |
| | virus (EBV) | | | | |
| KSEP-033 | Staphylococcus | 10 | 8 | 0.00042% | −0.1281 |
| | haemolyticus | | | | |

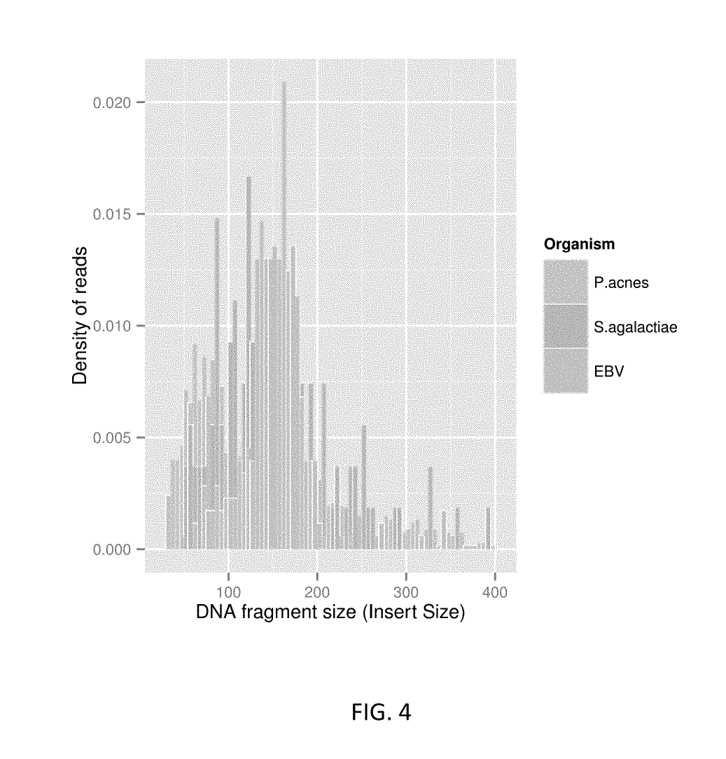

FIG. 4 shows an example of DNA fragment sizes of organisms in three samples. The inventors sought to enrich the “signal to noise ratio” in the data by increasing sensitivity of the analysis to the non-human cfDNA. As discussed above, microbial cfDNA found in plasma can be smaller in base pair length than nuclear DNA found in plasma. Increasing the “signal to noise ratio” may comprise increasing the ratio of non-human, microbial, or pathogen DNA as compared to human DNA in a sample or in a data set, such as in a set of sequencing reads from a sample. A sample, such as a plasma sample or other biological sample, may be obtained from a human. The cfDNA may be extracted from the sample. The extracted cfDNA may comprise human DNA and non-human DNA. In detecting and characterizing the non-human DNA in the extracted cfDNA, this disclosure provides a method for enriching the non-human cfDNA using size-selection, prior to or after sequencing the cfDNA and/or building the WGS libraries. The size selection may use a size threshold associated with human cfDNA or non-human cfDNA. For example, the size threshold may be based on a DNA fragment length of human cfDNA or an average DNA fragment length of human cfDNA. In various embodiments, the method may select a subset of cfDNA fragments which are shorter in length than average human cfDNA. For example, the cfDNA may be filtered for fragment lengths of 160 bp or less, or less than 166 bp, or less than 160 bp, or less than 150 bp, or less than 140 bp, or less than 130 bp prior to or during analysis of the sequencing reads. In various embodiments, the desired subset contains cfDNA fragments having a DNA fragment length of between 20-160 bp, or between 20-150 bp, or between 20-140 bp, or between 20-130 bp, or between 20-120 bp, or between 20-110, bp, or between 20-100 bp, or between 30-160 bp, or between 30-150 bp, or between 30-140 bp, or between 30-130 bp, or between 30-120 bp, or between 30-110 bp, or between 30-100 bp. or between 40-160 bp, or between 40-150 bp, or between 40-140 bp, or between 40-130 bp, or between 40-120 bp, or between 40-110 bp, or between 40-100 bp, or between 50-170 bp, or between 50-160 bp, or between 50-150 bp.

FIG. 4 shows the density of reads per DNA fragment size for the organisms found in a patient infected with Propionibacterium acnes (P. acnes), a patient infected with Streptococcus agalactiae (S. agalactiae), and a patient infected with Epstein-Barr virus (EBV). The EBV viral DNA is expected to be similar in length to human nucleosome fragments, averaging around 160 base pairs (bp) in length. The bacterial DNA fragment size (length) is less than the viral DNA fragment size, and is less than human DNA fragment size, which is the basis for the size-selection methods disclosed herein. As shown in FIG. 4, the average length of P. acnes DNA fragments is less than about 160 bp. Similarly, the average length of S. agalactiae DNA fragments is less than about 160 bp. By analyzing sequencing reads having a DNA fragment length (or read length) of 160 bp or less, or less than 166 bp, or less than 160 bp, or less than 150 bp, or less than 140 bp, the sequencing data is enriched for non-human cfDNA, thereby increasing the sensitivity of the method to microbial and/or pathogenic DNA.

The method of enriching non-human cfDNA within a sample from a human subject may comprise selecting a subset of the extracted cfDNA based on the size or fragment length of the cfDNA being less than the size threshold, i.e., less than an average fragment length of human cfDNA. Because the selected subset from the extracted cfDNA excludes longer cfDNA fragments, which are more likely to be human cfDNA, the subset has enriched non-human cfDNA. Stated differently, the size selection step enriches the ratio of non-human cfDNA to human cfDNA within the subset as compared to the ratio of non-human cfDNA to human cfDNA within the original set of extracted cfDNA. In various embodiments, the cfDNA fragments having a length of greater than 160 bp, or greater than 150 bp, or greater than 140 bp are excluded from the subset, thereby excluding human cfDNA from the subset.

To implement the size-selection approach, plasma cfDNA whole genome sequencing was used for the plasma samples of the three (3) patients, i.e., human samples, with blood-culture positive results. Using DNA fragment size selection, fewer reads can be used to successfully identify microorganisms present in cfDNA from plasma. For example, to obtain the results in TABLE 3, 14-20 million reads per sample were used. In 2 of 3 samples, size selection used to enrich the sequencing data resulted in a 10-fold enrichment in relative levels of microbial cfDNA. In the third sample, size selection used to enrich the data resulted in a 100-fold enrichment in relative levels of microbial DNA. TABLE 3 shows the results before and after size selection was used to enrich the sensitivity of the results. Without the disclosed method, the percent of non-human, microbial, or pathogen cfDNA within a sample may be difficult to detect and/or characterize, because the percentage or concentration of non-human cfDNA within the sample is low compared to the percentage or concentration of human cfDNA in the sample. By enriching the non-human cfDNA (“signal”) within the results, the sensitivity of the detection and/or characterization of the non-human cfDNA is improved.

TABLE 3
Results of Size Selection to Enrich Signal

| | | % Species | % Species | Z-score | Z-score |
|---|---|---|---|---|---|
| | Organism on | before Size | after Size | before Size | after Size |
| Patient ID | Culture | Selection | Selection | Selection | Selection |
| KSEP-013 | E. coli | 0.004% | 0.041% | −0.53 | −0.4720 |
| KSEP-020 | Group B | 0.04% | 0.61% | 5.66 | 88.15 |
| | Streptococcus | | | | |
| — | Epstein-Barr | 0.13% | 0.98% | 1.21 | 12.18 |
| | virus (EBV) | | | | |
| KSEP-033 | Staphylococcus | 0.00042% | 0.023% | −0.13 | 5.59 |
| | haemolyticus | | | | |

In patient KSEP-013, the percent of E. coli species found within the classified reads increased from 0.004% to 0.041% after size selection was applied to the reads, resulting in more than a 10-fold enrichment in the relative level of E. coli cfDNA. In patient KSEP-020, the percent of Group B Streptococcus species found within the classified reads increased from 0.04% to 0.061% after size selection was applied to the reads, resulting in a 10-fold enrichment in the relative level of Group B Streptococcus cfDNA. In patient KSEP-033, the percent of Staphylococcus haemolyticus species found within the classified reads increased from 0.00042% to 0.023% after size selection was applied to the reads, resulting in a 100-fold enrichment in the relative level of Staphylococcus haemolyticus cfDNA. EBV showed approximately a 5-fold enrichment after size selection. The enrichment of EBV was expected to be lower than the bacterial enrichment using size-selection, because EBV cfDNA fragment size is larger than bacterial cfDNA fragment size in plasma.

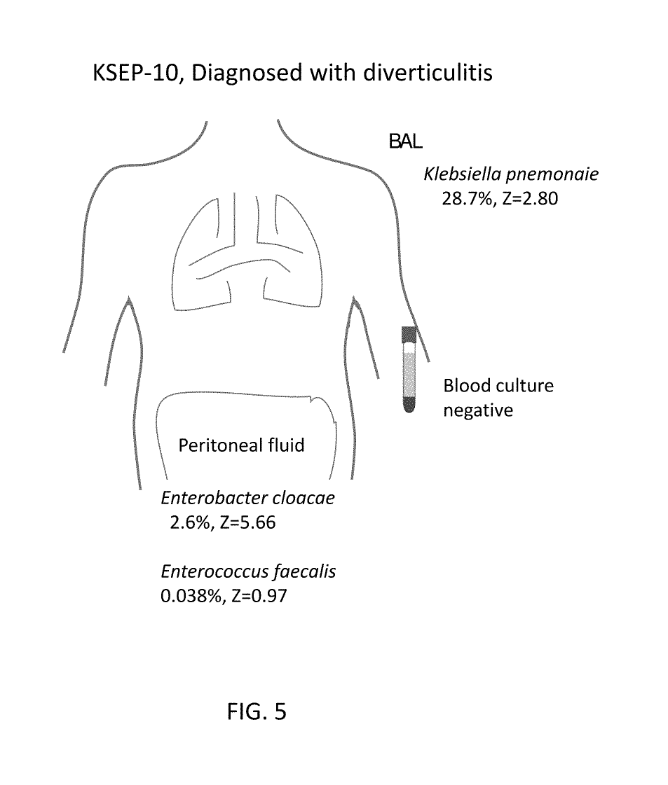

FIG. 5 shows a notable result of the present methods, where WGS of the plasma cfDNA detected pathogens in the subject's blood plasma, while the conventional method of culturing the blood failed to detect the pathogens. In patient KSEP-10, the blood culture was negative. However, the broncheo-alveolar lavage (BAL) culture for patient KSEP-10 was positive for Klebsiella pnemonaie, and the peritoneal fluid culture for patient KSEP-10 was positive for Enterobacter cloacae and Enterococcus faecalis. Whole genome plasma DNA sequencing found 28.7% Klebsiella pnemonaie within the classified reads after size selection, with a Z-score of 2.80. Whole genome plasma DNA sequencing found 2.8% Enterobacter cloacae within the classified reads after size selection, with a Z-score of 5.66. Whole genome plasma DNA sequencing found 0.038% Enterococcus faecalis within the classified reads after size selection, with a Z-score of 0.97. The results show that even where a blood culture fails to detect pathogens in other body cavities, the disclosed methods for whole genome plasma DNA sequencing is able to detect the pathogens in blood plasma.

Other results showed that whole genome plasma DNA sequencing is able to detect microorganisms which are undetectable in other cultures. In another patient, a BAL culture was positive for Citrobacter koseri, a rare pathogen, while the patient's blood culture was negative for Citrobacter koseri. Whole genome plasma DNA sequencing found 10 species-specific reads (0.23%, Z-score=5.66). None of the sequencing data from the 29 other patients in this study showed Citrobacter koseri reads.

In two patients KSEP-019 and KSEP-042, whole genome plasma DNA sequencing found E. coli, a more common pathogen that does not always cause infection. Patient KSEP-019 had a bedsore wound, which had a positive culture result when deep culturing was performed. The whole genome plasma DNA sequencing of the KSEP-019 day 0 plasma sample had a Z-score of 4.57 for E. coli. A blood culture for patient KSEP-042 was negative for E. coli. Whole genome plasma DNA sequencing of patient KSEP-042 found 2.3% E. coli with a Z-score of 2.88.

In one patient (KSEP-021) with necrotizing pancreatitis, the blood culture and BAL culture were negative. After three days of antibiotics, the patient underwent surgery, and the necrotic tissue was cultured. The culture was positive for Klebsiella pneumonia. The co-incident plasma sample taken on day 0 was whole genome sequenced. Whole genome plasma DNA sequencing found 20,090 species-specific reads (47.7%, Z-score=4.83) for Klebsiella pneumonia. The data shows that the presently disclosed method was able to detect the pathogen species.

### CONCLUSION

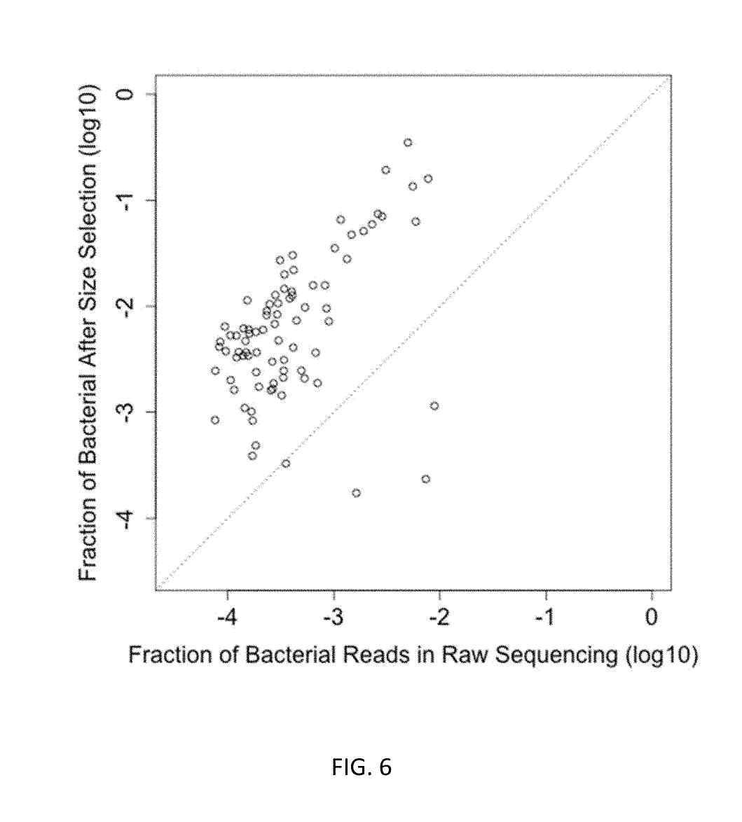

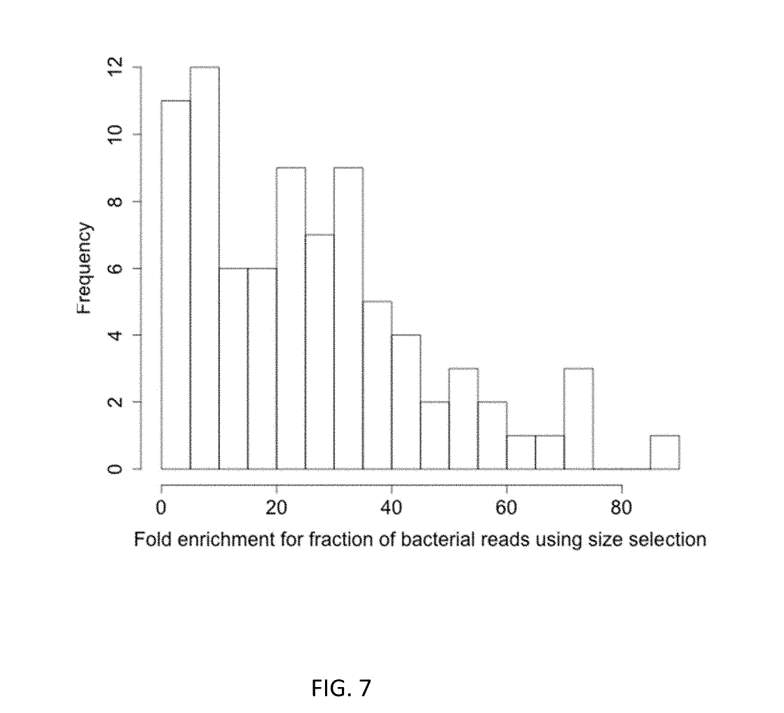

FIG. 6 compares the fraction of bacterial reads from raw sequencing data to the fraction of bacterial reads after size selection is applied to the sequencing data. FIG. 7 shows the frequency of enrichment fold values after size-selection. Size selection increased the fraction of sequencing reads that were successfully classified as bacterial by a median of 24.7 fold. In 82 plasma samples from 30 patients sequenced before and after size selection, we found a median 24.7 fold enrichment in fraction of sequencing reads classified as bacterial.

The results of this study show cfDNA from pathogens of sepsis is detectable in plasma DNA. WGS and outlier detection can potentially identify etiology of sepsis, particularly with respect to rare pathogens. Direct sequencing of bacterial cfDNA in plasma is feasible and may allow rapid identification of pathogens in patients with sepsis. On-going efforts are focused on refinement of informatics approaches and enrichment of non-human DNA in plasma samples to increase assay accuracy and reduce cost of sequencing.

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of diagnosing a pathogen in a plasma sample, the method comprising the steps of:
obtaining the plasma sample from a subject suspected of having the pathogen;
extracting cell-free DNA (cfDNA) from the plasma sample;
selecting a subset of the cfDNA based on the size of the cfDNA;
performing whole genome sequencing on the subset of the cfDNA to obtain sequencing data;
assigning the sequencing data to a candidate pathogen DNA; and
determining a presence of the pathogen in the plasma sample.

2. The method of claim 1, wherein the subset of the cfDNA is smaller in length than cfDNA excluded from the subset.

3. The method of claim 1, wherein selecting the subset of the cfDNA further comprises:
determining a size threshold associated with human cfDNA; and
selecting the subset of the cfDNA based on the size of cfDNA in the subset being below the size threshold.

4. The method of claim 4, wherein the size threshold comprises a DNA fragment length of 160 base pairs, or 150 base pairs, or 140 base pairs.

5. A method of detecting a microbe in a plasma sample, the method comprising the steps of:
obtaining the plasma sample from a subject;
extracting cell-free DNA (cfDNA) from the plasma sample, wherein the extracted cfDNA comprises human cfDNA and non-human cfDNA;
determining a fragment length threshold associated with human cfDNA;
performing whole genome sequencing on the extracted cfDNA to obtain sequencing data for the human cfDNA and the non-human cfDNA;
selecting a subset of the sequencing data based on the subset having a sequencing read length below the fragment length threshold;
assigning the subset of the sequencing data to a candidate microbe DNA; and
determining a presence of the microbe in the plasma sample.

6. The method of claim 5, wherein the subset comprises a greater ratio of non-human cfDNA to human cfDNA than the extracted cfDNA.

7. The method of claim 5, wherein selecting the sequencing data for the non-human cfDNA further comprises excluding the sequencing data for the human cfDNA

8. The method of claim 5, wherein the fragment length threshold is 160 base pairs, or 150 base pairs, or 140 base pairs.

9. A method of enriching non-human cfDNA within a blood sample from a human subject, the method comprising the steps of:
obtaining the blood sample from the human subject;
extracting cell-free DNA (cfDNA) from the blood sample to obtain extracted cfDNA, wherein the extracted cfDNA comprises human cfDNA and non-human cfDNA;
determining a size threshold associated with human cfDNA; and
selecting a subset of the extracted cfDNA based on the subset having a size below the size threshold, wherein the subset comprises a greater ratio of non-human cfDNA to human cfDNA than the extracted cfDNA.

10. The method of claim 9, further comprising:
performing whole genome sequencing on the subset of the extracted cfDNA to obtain sequencing data; and
assigning the sequencing data to a non-human candidate DNA.

11. The method of claim 9, further comprising:
performing whole genome sequencing on the extracted cfDNA to obtain sequencing data for the human cfDNA and the non-human cfDNA;
selecting the sequencing data for the non-human cfDNA; and
aligning the sequencing data for the non-human cfDNA with non-human candidate DNA to identify a microbial origin of the non-human cfDNA.

12. The method of claim 11, wherein selecting the sequencing data for the non-human cfDNA further comprises excluding the sequencing data for the human cfDNA.

13. The method of claim 11, wherein selecting the sequencing data for the non-human cfDNA further comprises selecting the sequencing data based on the size threshold.

14. The method of claim 13, wherein the size threshold comprises a DNA fragment length of 160 base pairs, or 150 base pairs, or 140 base pairs.

15. The method of claim 8, wherein the blood sample comprises a plasma sample.
